(19) United States
(12) Patent Application Publication
(10) Pub. No.: US 2021/0353618 A1
(43) Pub. Date: Nov. 18, 2021

(54) CROSS-LINKED SUPRAMOLECULAR NANOPARTICLES FOR CONTROLLED RELEASE OF ANTIFUNGAL DRUGS AND STEROIDS - A NEW THERAPEUTIC APPROACH FOR ONYCHOMYCOSIS AND KELOID

(71) Applicant: The Regents of the University of California, Oakland, CA (US)

(72) Inventors: Hsian-Rong Tseng, Los Angeles, CA (US); Yazhen Zhu, Los Angeles, CA (US)

(73) Assignee: The Regents of the University of California, Oakland, CA (US)

(21) Appl. No.: 17/290,605

(22) PCT Filed: Nov. 1, 2019
(86) PCT No.: PCT/US2019/059367

§ 371 Date: Apr. 30, 2021

Related U.S. Application Data
(60) Provisional application No. 62/754,657, filed on Nov. 2, 2018.

(51) Int. Cl. A61K 31/496; A61K 47/64; A61K 9/51; A61K 47/69; A61K 47/60; A61K 49/00; A61K 9/00; A61K 47/54

(52) U.S. Cl. CPC A61K 31/496; A61K 47/645; A61K 9/51; A61K 47/6925; A61K 47/54; A61K 49/0093; A61K 9/0019; A61K 47/547; A61K 47/60

(57) ABSTRACT

Compositions for delivering a drug to a subject having: a plurality of self-assembled supramolecular nanoparticles (SMNPs), each of the plurality of self-assembled supramolecular nanoparticles (SMNPs) having: a plurality of binding components, each having a plurality of binding regions; a plurality of cores that are suitable to at least provide some mechanical structure to the plurality of self-assembled supramolecular nanoparticles (SMNPs), the plurality of cores comprising at least one core binding element adapted to bind to the binding regions to form a first inclusion complex; a plurality of terminating components, each having a single terminating binding element that binds to remaining binding regions of one of said plurality of binding components by forming a second inclusion complex; the drug; and a reporter agent, and methods of use thereof.

### CROSS-REFERENCE OF RELATED APPLICATIONS

This application claims priority to U.S. Provisional Application No. 62/754,657 filed Nov. 2, 2018; the entire contents of which are hereby incorporated by reference.

### GOVERNMENT SUPPORT CLAUSE

This invention was made with government support under Grant Number EB016270, awarded by the National Institutes of Health. The government has certain rights in the invention.

### BACKGROUND

### 1. Technical Field

Aspects of the invention relate to compositions, systems and methods for delivering a drug to a subject via the use of a plurality of self-assembled supramolecular nanoparticles (SMNPs).

### 2. Discussion of Related Art

Onychomycosis is a progressive, contagious, and recurring fungal infection of the nail apparatus, which has been considered a “clinically stubborn disease” with high prevalence (approximately 10-12% of the general U.S. population) and low cure rates.¹⁻⁴ Aside from mere cosmetic concerns, fungal nail infections can also cause severe health problems, such as high risk of contamination with other nails in a same patient or other susceptible individuals; serious complications in diabetic or elderly people;^{5,6} recurrent cellulitis and thrombophlebitis;⁷ and significantly reduced quality of life.⁸

A large variety of therapeutic approaches⁸⁻¹², including oral administration, topical creams, laser-based treatment, and combined treatments have been developed to treat onychomycosis. Oral administration based on approved anti-fungal drugs, e.g., allylamines, azoles, morpholines, benzoxaboroles, and hydroxypyridinones, have been widely used.^{2, 13} Since the oral administration only achieve temporary effective drug concentration at the fungal infection sites,¹⁴ a prolonged high-dose treatment is required in order to sustain therapeutic efficacy at the sites of fungal infection. As a result, systemic side effects such as liver toxicity, potential drug reactions, and bioavailability problems^{9,14} have limited clinical utility of oral drugs. Although topical creams exhibit minimum systemic toxicity and off-target effects, this approach has especially low cure rates¹⁵⁻¹⁷ due to the nail plate acting as a barrier to the infected site. In contrast to the oral and topical methods, laser-based treatments use direct heat treatment to thermally eradicate nail fungus.^{18,19} Due to technical challenge to precisely deliver laser pulses to the fungal infected sites, most laser systems employ nonspecific bulk heating, presenting possibility of damage to the surrounding healthy tissue. The ineffectiveness and complexity of the existing therapeutic approaches⁹ highlight an unmet need for a new anti-fungal therapeutic approach, capable of sustainably eradicating nail fungus. An ideal therapeutic approach for onychomycosis would have the advantages of (i) ability to introduce anti-fungal drugs directly to the infected site; (ii) finite intradermal sustainable release to maintain effective drug levels over prolonged time; (iii) a reporter system for monitoring maintenance of drug level; and (iv) minimum level of inflammatory responses at or around the fungal infection sites.

### INCORPORATION BY REFERENCE

All publications and patent applications identified herein are incorporated by reference in their entirety and to the same extent as if each individual publication or patent application was specifically and individually indicated to be incorporated by reference.

### SUMMARY

An embodiment of the invention relates to a composition for delivering a drug to a subject having: a plurality of self-assembled supramolecular nanoparticles (SMNPs), each of the plurality of self-assembled supramolecular nanoparticles (SMNPs) having: a plurality of binding components, each having a plurality of binding regions; a plurality of cores that are suitable to at least provide some mechanical structure to the plurality of self-assembled supramolecular nanoparticles (SMNPs), the plurality of cores comprising at least one core binding element adapted to bind to the binding regions to form a first inclusion complex; a plurality of terminating components, each having a single terminating binding element that binds to remaining binding regions of one of said plurality of binding components by forming a second inclusion complex; the drug; and a reporter agent. In such an embodiment, the plurality of binding components and the plurality of cores self-assemble when brought into contact to form the plurality of self-assembled supramolecular nanoparticles (SMNPs), the plurality of terminating components act to occupy the remaining binding regions of the plurality of binding components, and the plurality of terminating components are present in a sufficient quantity relative to the plurality of binding regions of the plurality of binding components to terminate further binding, thereby forming a discrete particle, the drug is encapsulated within each of the plurality of self-assembled supramolecular nanoparticles (SMNPs), the reporter agent is encapsulated within each of the plurality of supramolecular nanoparticles (SMNPs), the plurality of cores and the plurality of binding components are present in a percent mass (w/w) ratio of between 0.25:1 and 2.5:1, and each of the plurality of self-assembled supramolecular nanoparticles (SMNPs) has a diameter of between 240 nanometers and 730 nanometers.

An embodiment of the invention relates to a method for making a composition comprising a plurality of self-assembled supramolecular nanoparticles (SMNPs) for delivering a drug to a subject including: providing a first solution comprising a plurality of binding components, each having a plurality of binding regions; providing a second solution comprising a plurality of cores that are suitable to at least provide some mechanical structure to the plurality of self-assembled supramolecular nanoparticles (SMNPs), the plurality of cores comprising at least one core binding element adapted to bind to the binding regions to form a first inclusion complex; providing a third solution comprising a plurality of terminating components, each having a single terminating binding element that binds to remaining binding regions of one of said plurality of binding components by forming a second inclusion complex; providing a fourth solution comprising the drug; providing a fifth solution comprising a reporter agent; and mixing the first solution, the second solution, the third solution, the fourth solution, and the fifth solution. In such an embodiment, the mixing brings into contact the plurality of binding components and the plurality of cores such that the plurality of binding components and the plurality of cores self-assemble to form the plurality of self-assembled supramolecular nanoparticles (SMNPs), and such that the drug and the reporter agent are encapsulated within each of the plurality of self-assembled supramolecular nanoparticles (SMNPs), the plurality of terminating components act to occupy the remaining binding regions of the plurality of binding components, and the plurality of terminating components are present in a sufficient quantity relative to the plurality of binding regions of the plurality of binding components to terminate further binding, thereby forming a discrete particle, the plurality of cores and the plurality of binding components are present in a percent mass (w/w) ratio of between 0.25:1 and 2.5:1, and each of the plurality of self-assembled supramolecular nanoparticles (SMNPs) has a diameter of between 240 nanometers and 730 nanometers.

An embodiment of the invention relates to a method for delivering a drug to a subject including: penetrating an epidermis tissue layer of the subject such that an accession point to an underlying dermis layer in the subject is created; and delivering a plurality of self-assembled supramolecular nanoparticles (SMNPs) to the underlying dermis layer in the subject through the accession point. In such an embodiment, each of the plurality of self-assembled supramolecular nanoparticles (SMNPs) have: a plurality of binding components, each having a plurality of binding regions; a plurality of cores that are suitable to at least provide some mechanical structure to the plurality of self-assembled supramolecular nanoparticles (SMNPs), the plurality of cores comprising at least one core binding element adapted to bind to the binding regions to form a first inclusion complex; a plurality of terminating components, each having a single terminating binding element that binds to remaining binding regions of one of said plurality of binding components by forming a second inclusion complex; the drug; and a reporter agent. In such an embodiment, the plurality of binding components and the plurality of cores self-assemble when brought into contact to form the plurality of self-assembled supramolecular nanoparticles (SMNPs), the plurality of terminating components act to occupy the remaining binding regions of the plurality of binding components, and the plurality of terminating components are present in a sufficient quantity relative to the plurality of binding regions of the plurality of binding components to terminate further binding, thereby forming a discrete particle, the drug is encapsulated within each of the plurality of self-assembled supramolecular nanoparticles (SMNPs), the reporter agent is encapsulated within each of the plurality of supramolecular nanoparticles (SMNPs), the wherein the plurality of cores and the plurality of binding components are present in a percent mass (w/w) ratio of between 0.25:1 and 2.5:1, and each of the plurality of self-assembled supramolecular nanoparticles (SMNPs) has a diameter of between 240 nanometers and 730 nanometers.

### BRIEF DESCRIPTION OF THE DRAWINGS

FIGS. 1A and 1B are illustrations showing formation of ketoconazole-encapsulated cross-linked fluorescent supramolecular nanoparticles (KTZCc-FSMNPs), and their use as a therapeutic approach for treating onychomycosis according to an embodiment of the invention;

FIGS. 2A-2D are graphs and electron microscope images showing characterization of size-controlled ketoconazole-encapsulated cross-linked fluorescent supramolecular nanoparticles (KTZcFSMNPs) according to an embodiment of the invention;

FIGS. 3A-3E are schematics, images, and data graphs showing the formation and characterization of micrometer-sized ketoconazole-encapsulated cross-linked fluorescent supramolecular nanoparticles (KTZCc-FSMNPs) according to an embodiment of the invention;

FIGS. 4A and 4B are images and a data graph showing the correlation between fluorescence intensity and residual ketoconazole (KTZ) concentration after tattoo deposition of 4800 nm KTZ-encapsulated cross-linked fluorescent supramolecular nanoparticles (KTZCc-FSMNPs) according to an embodiment of the invention;

FIGS. 5A-5F are images, data graphs, and stained skin sections from an intradermal retention study of ketoconazole-encapsulated cross-linked fluorescent supramolecular nanoparticles (KTZ⊂c-FSMNPs) according to an embodiment of the invention.

### DETAILED DESCRIPTION

Existing approaches for treating onychomycosis demonstrate limited success since the commonly used oral administration and topical cream only achieve temporary effective drug concentration at the fungal infection sites. An ideal therapeutic approach for onychomycosis should have (i) the ability to introduce antifungal drugs directly to the infected sites; (ii) finite intradermal sustainable release to maintain effective drug levels over prolonged time; (iii) a reporter system for monitoring maintenance of drug level; and (iv) minimum level of inflammatory responses at or around the fungal infection sites. Embodiments disclosed herein include and accomplish all four of these points. An embodiment comprises ketoconazole-encapsulated cross-linked fluorescent supramolecular nanoparticles (KTZ⊂c-FSMNPs) as an intradermal controlled release solution for treating onychomycosis. An embodiment comprises a two-step synthetic approach adopted to prepare a variety of KTZ⊂c-FSMNPs. In some embodiments, 4800 nm KTZ⊂c-FSMNPs exhibited high KTZ encapsulation efficiency/capacity, optimal fluorescent property, and sustained KTZ release profile.

An embodiment of the invention relates to a composition for delivering a drug to a subject having: a plurality of self-assembled supramolecular nanoparticles (SMNPs), each of the plurality of self-assembled supramolecular nanoparticles (SMNPs) having: a plurality of binding components, each having a plurality of binding regions; a plurality of cores that are suitable to at least provide some mechanical structure to the plurality of self-assembled supramolecular nanoparticles (SMNPs), the plurality of cores comprising at least one core binding element adapted to bind to the binding regions to form a first inclusion complex; a plurality of terminating components, each having a single terminating binding element that binds to remaining binding regions of one of said plurality of binding components by forming a second inclusion complex; the drug; and a reporter agent. In such an embodiment, the plurality of binding components and the plurality of cores self-assemble when brought into contact to form the plurality of self-assembled supramolecular nanoparticles (SMNPs), the plurality of terminating components act to occupy the remaining binding regions of the plurality of binding components, and the plurality of terminating components are present in a sufficient quantity relative to the plurality of binding regions of the plurality of binding components to terminate further binding, thereby forming a discrete particle, the drug is encapsulated within each of the plurality of self-assembled supramolecular nanoparticles (SMNPs), the reporter agent is encapsulated within each of the plurality of supramolecular nanoparticles (SMNPs), the plurality of cores and the plurality of binding components are present in a percent mass (w/w) ratio of between 0.25:1 and 2.5:1, and each of the plurality of self-assembled supramolecular nanoparticles (SMNPs) has a diameter of between 240 nanometers and 730 nanometers.

An embodiment of the invention relates to the composition above, where the plurality of binding components comprises polythylenimine, poly(L-lysine), or poly(β-amino ester).

An embodiment of the invention relates to the composition above, where the plurality of binding regions comprises beta-cyclodextrin, alpha-cyclodextrin, gamma-cyclodextrin, cucurbituril or calixarene.

An embodiment of the invention relates to the composition above, where the plurality of cores comprises polyamidoamine dendrimers, poly(prophylenimine) (PPI) dendrimer, triazine dendrimer, carbosilane dendrimer, poly(ether imine) (PETIM) dendrimer or phosphorus dendrimer.

An embodiment of the invention relates to the composition above, where the at least one core binding element comprises adamantanamine, azobenzene, ferrocene or anthracene.

An embodiment of the invention relates to the composition above, where the plurality of terminating components comprises polyethylene glycol (PEG) or poly(propylene glycol) (PGG).

An embodiment of the invention relates to the composition above, where the single terminating binding element comprises adamantanamine, azobenzene, ferrocene or anthracene.

An embodiment of the invention relates to the composition above, where the plurality of self-assembled supramolecular nanoparticles (SMNPs) are configured to release the drug into the subject over a period of time.

An embodiment of the invention relates to the composition above, where the period of time is at least 14 days in length.

An embodiment of the invention relates to the composition above, where each of the plurality of SMNPs are cross-linked to one or more of the plurality of SMNPs such that a cross-linked network of SMNPs is formed.

An embodiment of the invention relates to the composition above, where the cross-linked network of SMNPs has a maximum spatial dimension of between 2020 nanometers and 5030 nanometers.

An embodiment of the invention relates to the composition above, where the drug is selected from the group consisting of an anti-viral drug, an anti-bacterial drug, and an anti-fungal drug.

An embodiment of the invention relates to the composition above, where the reporter agent is a fluorescent probe.

An embodiment of the invention relates to the composition above, where the plurality of self-assembled supramolecular nanoparticles (SMNPs) are configured to release the drug at a first rate of release, where the plurality of self-assembled supramolecular nanoparticles (SMNPs) are configured to release the reporter agent at a second rate of release, and where the second rate of release is correlated with first rate of release.

An embodiment of the invention relates to a method for making a composition comprising a plurality of self-assembled supramolecular nanoparticles (SMNPs) for delivering a drug to a subject including: providing a first solution comprising a plurality of binding components, each having a plurality of binding regions; providing a second solution comprising a plurality of cores that are suitable to at least provide some mechanical structure to the plurality of self-assembled supramolecular nanoparticles (SMNPs), the plurality of cores comprising at least one core binding element adapted to bind to the binding regions to form a first inclusion complex; providing a third solution comprising a plurality of terminating components, each having a single terminating binding element that binds to remaining binding regions of one of said plurality of binding components by forming a second inclusion complex; providing a fourth solution comprising the drug; providing a fifth solution comprising a reporter agent; and mixing the first solution, the second solution, the third solution, the fourth solution, and the fifth solution. In such an embodiment, the mixing brings into contact the plurality of binding components and the plurality of cores such that the plurality of binding components and the plurality of cores self-assemble to form the plurality of self-assembled supramolecular nanoparticles (SMNPs), and such that the drug and the reporter agent are encapsulated within each of the plurality of self-assembled supramolecular nanoparticles (SMNPs), the plurality of terminating components act to occupy the remaining binding regions of the plurality of binding components, and the plurality of terminating components are present in a sufficient quantity relative to the plurality of binding regions of the plurality of binding components to terminate further binding, thereby forming a discrete particle, the plurality of cores and the plurality of binding components are present in a percent mass (w/w) ratio of between 0.25:1 and 2.5:1, and each of the plurality of self-assembled supramolecular nanoparticles (SMNPs) has a diameter of between 240 nanometers and 730 nanometers.

An embodiment of the invention relates to the method above, where the plurality of binding components comprises polythylenimine, poly(L-lysine), or poly(-amino ester).

An embodiment of the invention relates to the method above, where the plurality of binding regions comprises beta-cyclodextrin, alpha-cyclodextrin, gamma-cyclodextrin, cucurbituril or calixarene.

An embodiment of the invention relates to the method above, where the plurality of cores comprises polyamidoamine dendrimers, poly(prophylenimine) (PPI) dendrimer, triazine dendrimer, carbosilane dendrimer, poly(ether imine) (PETIM) dendrimer or phosphorus dendrimer.

An embodiment of the invention relates to the method above, where the at least one core binding element comprises adamantanamine, azobenzene, ferrocene or anthracene.

An embodiment of the invention relates to the method above, where the plurality of terminating components comprises polyethylene glycol (PEG) or poly(propylene glycol) (PGG).

An embodiment of the invention relates to the method above, where the single terminating binding element comprises adamantanamine, azobenzene, ferrocene or anthracene.

An embodiment of the invention relates to the method above, where the plurality of self-assembled supramolecular nanoparticles (SMNPs) are configured to release the drug into the subject over a period of time.

An embodiment of the invention relates to the method above, where the period of time is at least 14 days in length.

An embodiment of the invention relates to the method above, further including cross-linking the each of the plurality of self-assembled supramolecular nanoparticles (SMNPs) to one or more of the plurality of self-assembled supramolecular nanoparticles (SMNPs) such that a cross-linked network of self-assembled supramolecular nanoparticles (SMNPs) is formed.

An embodiment of the invention relates to the method above, where the cross-linked network of self-assembled supramolecular nanoparticles (SMNPs) has a maximum spatial dimension of between 2020 nanometers and 5030 nanometers.

An embodiment of the invention relates to the method above, where the drug is selected from the group consisting of an anti-viral drug, an anti-bacterial drug, and an anti-fungal drug.

An embodiment of the invention relates to the method above, where the reporter agent is a fluorescent probe.

An embodiment of the invention relates to the method above, where the plurality of self-assembled supramolecular nanoparticles (SMNPs) are configured to release the drug at a first rate of release, the plurality of self-assembled supramolecular nanoparticles (SMNPs) are configured to release the reporter agent at a second rate of release, and the second rate of release is correlated with first rate of release.

An embodiment of the invention relates to a method for delivering a drug to a subject including: penetrating an epidermis tissue layer of the subject such that an accession point to an underlying dermis layer in the subject is created; and delivering a plurality of self-assembled supramolecular nanoparticles (SMNPs) to the underlying dermis layer in the subject through the accession point. In such an embodiment, each of the plurality of self-assembled supramolecular nanoparticles (SMNPs) have: a plurality of binding components, each having a plurality of binding regions; a plurality of cores that are suitable to at least provide some mechanical structure to the plurality of self-assembled supramolecular nanoparticles (SMNPs), the plurality of cores comprising at least one core binding element adapted to bind to the binding regions to form a first inclusion complex; a plurality of terminating components, each having a single terminating binding element that binds to remaining binding regions of one of said plurality of binding components by forming a second inclusion complex; the drug; and a reporter agent. In such an embodiment, the plurality of binding components and the plurality of cores self-assemble when brought into contact to form the plurality of self-assembled supramolecular nanoparticles (SMNPs), the plurality of terminating components act to occupy the remaining binding regions of the plurality of binding components, and the plurality of terminating components are present in a sufficient quantity relative to the plurality of binding regions of the plurality of binding components to terminate further binding, thereby forming a discrete particle, the drug is encapsulated within each of the plurality of self-assembled supramolecular nanoparticles (SMNPs), the reporter agent is encapsulated within each of the plurality of supramolecular nanoparticles (SMNPs), the wherein the plurality of cores and the plurality of binding components are present in a percent mass (w/w) ratio of between 0.25:1 and 2.5:1, and each of the plurality of self-assembled supramolecular nanoparticles (SMNPs) has a diameter of between 240 nanometers and 730 nanometers.

An embodiment of the invention relates to the method above, where the plurality of binding components comprises polythylenimine, poly(L-lysine), or poly(β-amino ester).

An embodiment of the invention relates to the method above, where the plurality of binding regions comprises beta-cyclodextrin, alpha-cyclodextrin, gamma-cyclodextrin, cucurbituril or calixarene.

An embodiment of the invention relates to the method above, where the plurality of cores comprises polyamidoamine dendrimers, poly(prophylenimine) (PPI) dendrimer, triazine dendrimer, carbosilane dendrimer, poly(ether imine) (PETIM) dendrimer or phosphorus dendrimer.

An embodiment of the invention relates to the method above, where the at least one core binding element comprises adamantanamine, azobenzene, ferrocene or anthracene.

An embodiment of the invention relates to the method above, where the plurality of terminating components comprises polyethylene glycol (PEG) or poly(propylene glycol) (PGG).

An embodiment of the invention relates to the method above, where the single terminating binding element comprises adamantanamine, azobenzene, ferrocene or anthracene.

An embodiment of the invention relates to the method above, where the plurality of self-assembled supramolecular nanoparticles (SMNPs) are configured to release the drug into the subject over a period of time.

An embodiment of the invention relates to the method above, where the period of time is at least 14 days in length.

An embodiment of the invention relates to the method above, where each of the plurality of self-assembled SMNPs are cross-linked to one or more of the plurality of self-assembled SMNPs such that a cross-linked network of SMNPs is formed.

An embodiment of the invention relates to the method above, where the cross-linked network of SMNPs has a maximum spatial dimension of between 2020 nanometers and 5030 nanometers.

An embodiment of the invention relates to the method above, where the drug is selected from the group consisting of an anti-viral drug, an anti-bacterial drug, and an anti-fungal drug.

An embodiment of the invention relates to the method above, where the reporter agent is a fluorescent probe.

An embodiment of the invention relates to the method above, where the plurality of self-assembled supramolecular nanoparticles (SMNPs) are configured to release the drug at a first rate of release, the plurality of self-assembled supramolecular nanoparticles (SMNPs) are configured to release the reporter agent at a second rate of release, and the second rate of release is correlated with first rate of release.

Some aspects of the invention include supramolecular nanoparticles (SMNPs), having a plurality of binding components, each having a plurality of binding regions; a plurality of cores that are suitable to at least provide some mechanical structure to the plurality of self-assembled supramolecular nanoparticles (SMNPs), the plurality of cores comprising at least one core binding element adapted to bind to the binding regions to form a first inclusion complex; and a plurality of terminating components, each having a single terminating binding element that binds to remaining binding regions of one of said plurality of binding components by forming a second inclusion complex. SMNPs are described in in U.S. Pat. No. 9,845,237 and U.S. Patent Application Publication No. 2016/0000918, each of which is herein incorporated in its entirety by reference. The plurality of binding components, plurality of cores, and the plurality of terminating components self-assemble when brought into contact to form the supramolecular magnetic nanoparticle (SMNP).

The plurality of binding components, plurality a cores, and the plurality of terminating components bind to each other by one or more intermolecular forces. Examples of intermolecular forces include hydrophobic interactions, biomolecular interactions, hydrogen bonding interactions, π-π interactions, electrostatic interactions, dipole-dipole interactions, or van der Waals forces. Examples of biomolecular interactions include DNA hybridization, a protein-small molecule interaction (e.g. protein-substrate interaction (e.g. a streptavidin-biotin interaction) or protein-inhibitor interaction), an antibody-antigen interaction or a protein-protein interaction. Examples of other interactions include inclusion complexes or inclusion compounds, e.g. adamantane-β-cyclodextrin complexes or diazobenzene-α-cyclodextrin complexes. Generally, the intermolecular forces binding the components of the SMNP structure are not covalent bonds.

Some embodiments of the invention comprise a “reporter agent.” As used throughout, the term “reporter agent” refers to a molecule, compound, protein, etc. that is used to assess and/or monitor the delivery and/or release of a drug to a tissue. A reporter agent can be a naturally occurring agent, a synthetic agent, or a combination thereof. In some embodiments the reporter agent emits a fluorescent signal. In some embodiments, the rate of release of the reporter agent from a nanoparticle correlates with the rate of release of a drug from a nanoparticle.

Some embodiments of the invention include a method for delivering a nanoparticle carrying a drug and/or reporter agent to a tissue and include a step of penetrating the epidermis layer of the tissue. Methods of penetrating the epidermis layer of the tissue are apparent to one of ordinary skill in the art. In some embodiments, the nanoparticles are delivered using the same tool used to penetrate the epidermis layer of the tissue. In some embodiments, the epidermis layer is penetrated using a first tool to create an incision site, and the nanoparticles are delivered into the incision site using a separate tool.

The following describes some embodiments of the current invention more specifically. The general concepts of this invention are not limited to these particular embodiments.

### EXAMPLE

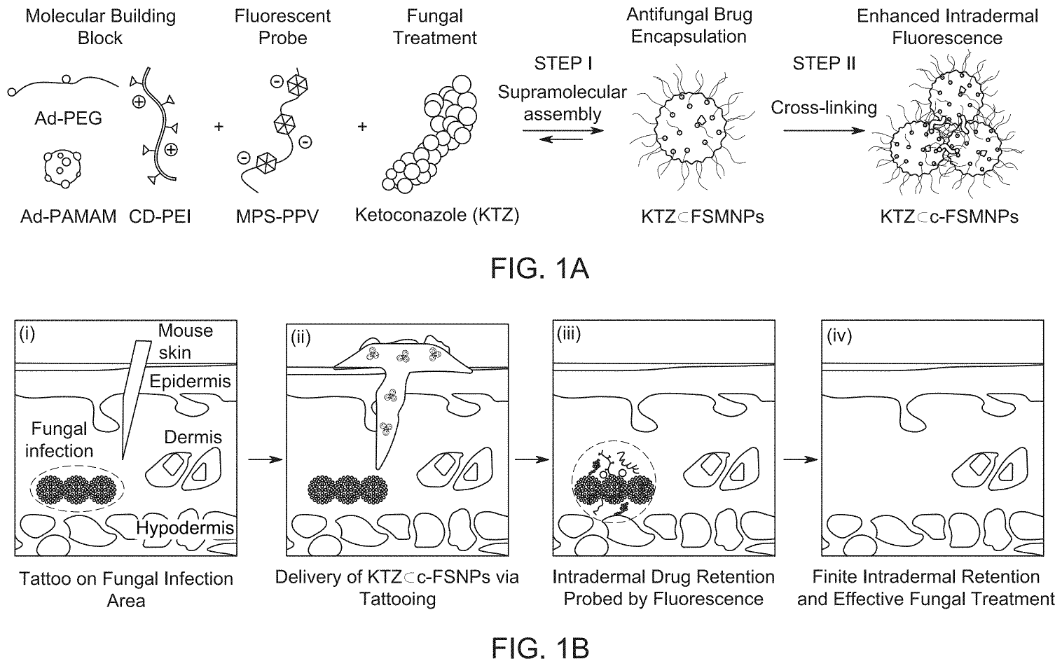

Previously, a convenient, flexible, and modular self-assembled synthetic approach^{20,21} for the preparation of supra-molecular nanoparticle (SMNP) vectors from a collection of molecular building blocks (i.e., Ad-PEG, Ad-PAMAM, and CD-PEI) through a multivalent molecular recognition between adamantane (Ad) and β-cyclodextrin (CD) motifs was demonstrated. Such a self-assembled synthetic strategy enables control over the sizes, surface chemistry, and payloads of SMNP vectors for both diagnostic and therapeutic applications.²²⁻²⁹ Using this technique, encapsulation of hydrophobic drug molecules (e.g., doxorubicin) into the SMNP vectors for in vivo cancer treatment was demonstrated.³⁰ Successful preparation of cross-linked fluorescent supramolecular nanoparticles (c-FSMNPs) by encapsulating a fluorescent conjugated polymer, that is, poly[5-methoxy-2-(3-propyloxysulfonate)-1,4-phenylenevinylene] potassium salt (MPS-PPV) into the SMNP vectors, followed by a cross-linking reaction was also demonstrated.³¹ It was shown that these c-FSMNPs exhibit enhanced photophysical properties, a finite intradermal retention, and bio-compatibility, making them a promising candidate as an ideal tattoo pigment. On the basis of past studies with SMNP vectors, the possible utility of c-FSMNPs as an ideal controlled release strategy to deliver a commonly used azole-based antifungal drug, ketoconazole (KTZ), intradermally, paving the way for implementing an onychomycosis treatment solution was explored. Ketoconazole is one of the most commonly used drugs for onychomycosis treatment through oral administration and topical application. As mentioned above, KTZ's treatment efficacy has been limited due to its insufficient local drug concentration at the disease sites.³² Ketoconazole-encapsulated cross-linked fluorescent supramolecular nanoparticles (KTZ⊂c-FSMNPs) can be prepared with the desired optical properties so as to enable in vivo controlled release performance by using a two-step synthetic approach (FIG. 1a). In the first step, KTZ⊂FSMNPs are obtained with controllable sizes by performing ratiometric mixing among KTZ, MPS-PPV, and the three SMNP molecular building blocks (i.e., Ad-PEG, Ad-PAMAM, and CD-PEI) using the supramolecular synthetic strategy.^{20,31} In the second step, a cross-linking reaction is employed on KTZ⊂FSMNPs to generate micrometer-sized KTZ⊂c-FSMNPs.

Here, it is disclosed that that KTZ⊂FSMNPs and KTZ⊂c-FSMNPs exhibited controllable sizes, high KTZ encapsulation efficiency and capacity, enhanced fluorescent properties, and KTZ controlled release profile. Using female nude mice as an animal model (Athymic Nude-Foxn1nu purchased from Envigo), KTZ⊂c-FSMNPs were introduced into intradermal spaces of the mice via a skin tattoo method (FIG. 1b). The intradermal retention properties of KTZ⊂c-FSMNPs were examined by in vivo fluorescent imaging for monitoring the time-dependent decay of KTZ⊂c-FSMNPs' fluorescent signals, high-performance liquid chromatography (HPLC) quantification of residual KTZ in skin tissues, harvested at different time points, (iii) matrix-assisted laser desorption/ionization mass spectrometry imaging (MALDI-MSI) for mapping the KTZ distribution in intradermal regions around the tattoo sites, and (iv) histology assessment on local inflammatory responses and biocompatibility. The time dependent in vivo fluorescent imaging and HPLC quantification suggested that 4800 nm KTZ⊂c-FSMNPs exhibited a prolonged retention time up to 14 days. Furthermore, the skin histology studies indicated minimum inflammatory responses to the tattooed KTZ⊂c-FSMNPs, demonstrating good biocompatibility. Such a finite intradermal retention and biocompatibility make them a promising candidate as a therapeutic approach for intradermal controlled release of antifungal drug to treat onychomycosis. In contrast to the existing microneedle (MN)-based intradermal delivery approaches,³³⁻³⁸ the disclosed tattoo-based delivery of KTZ⊂c-FSMNPs offers an alternative with advantages including convenient self-assembled synthesis, and well-controlled intradermal puncture/delivery depth.

FIGS. 1A and 1B are illustrations showing formation of ketoconazole-encapsulated cross-linked fluorescent supramolecular nanoparticles (KTZCc-FSMNPs), and their use as a therapeutic approach for treating onychomycosis. FIG. 1A shows a two-step synthetic approach employed for the preparation of KTZCc-FSMNPs: (Step I) supramolecular assembly of KTZ, MPS-PPV, and the three SMNP molecular building blocks (i.e., Ad-PEG, Ad-PAMAM, and CD-PEI) gives KTZ-encapsulated fluorescent supramolecular nanoparticles (KTZCFSMNPs); (Step II) cross-linking of KTZCFSMNPs yields micrometer-sized KTZCc-FSMNPs. FIG. 1b is a schematic illustration of intradermal deposition of KTZCc-FSMNPs via tattoo: (i) tattoo in the dermal layer of the mouse skin through poking with a commercial tattoo needle, (ii) introduction of the KTZCc-FSMNPs into the mouse skin, (iii) controlled release of KTZ at fungal infection sites with intradermal drug retention probed by fluorescence, and (iv) clearance of tattooed KTZCc-FSMNPs with a finite intradermal retention time

### Results and Discussion

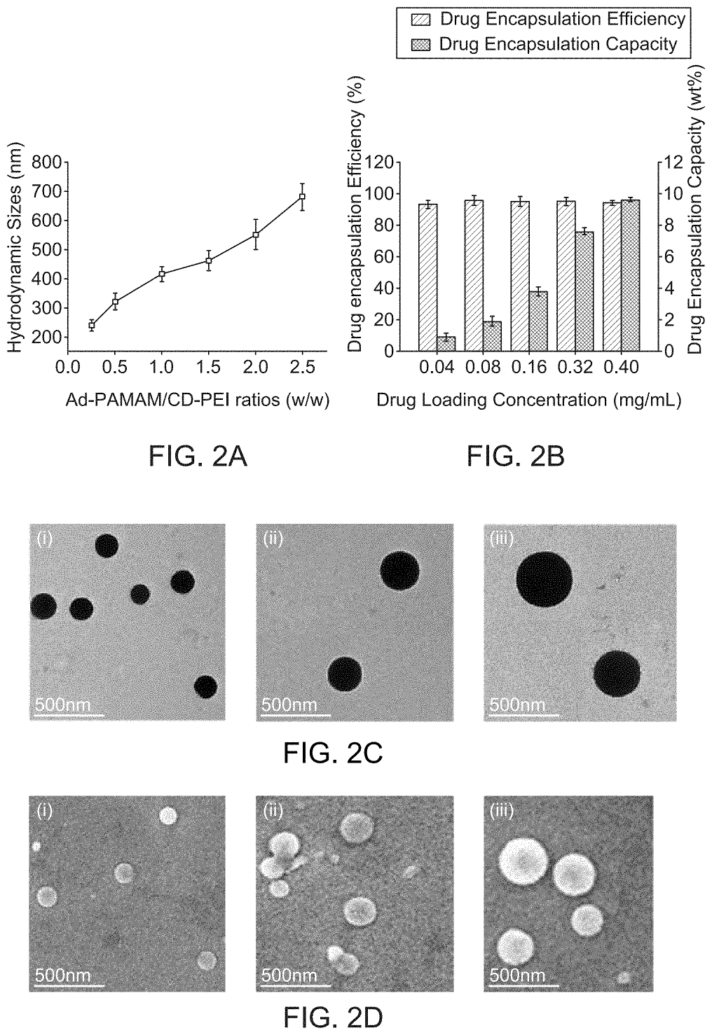

The supramolecular synthetic strategy^{20,31} was used to prepare size-controllable KTZ⊂FSMNPs by performing ratiometric mixing of KTZ, MPS-PPV, and the three SMNP molecular building blocks (i.e., Ad-PEG, Ad-PAMAM, and CD-PEI). KTZ and MPS-PPV were encapsulated into the intraparticular spaces of SMNP vectors according to the mechanisms observed for doxorubicincFSMNPs³⁰ and FSMNPs³¹ By keeping the concentrations of KTZ (0.16 mg/mL), MPS-PPV (0.12 mg/mL), Ad-PEG (1.84 mg/mL), and CD-PEI (0.04 mg/mL) constant, the weight ratios between Ad-PAMAM and CD-PEI (Ad-PAMAM/CD-PEI, w/w; 0.25:1, 0.5:1, 1.0:1, 1.5:1, 2.0:1 and 2.5:1) were altered to control the sizes of the resulting KTZCFSMNPs. Subsequently, dynamic light scattering (DLS) measurements were utilized to analyze hydrodynamic sizes of the freshly prepared KTZCFSMNPs. As shown in FIG. 2a, a collection of water-soluble KTZCFSMNPs with variable sizes ranging between 240 and 680 nm were obtained. Increasing the ratio of Ad-PAMAM/CD-PEI can increase the size of KTZCFSMNPs, which was consistent with prior studies.^{20,31} As expected, the tattooed FSMNPs exhibited a size-dependent intradermal retention time, which increased with increasing particle sizes. KTZ encapsulation efficiency and capacity of KTZCFSMNPs was further studied, at different KTZ concentrations (0.04 to 0.4 mg/mL) while keeping the Ad-PAMAM/CD-PEI mixing ratio constant (2.5:1). High-performance liquid chromatography (HPLC) was utilized to quantify the KTZ partition in both solution phase and KTZCFSMNPs, suggesting a KTZ encapsulation efficiency (^{˜}94%) across different formulation conditions. FIG. 2b summarizes that the KTZ encapsulation capacities varied between 0.92 and 9.4 wt % at different KTZ concentrations. While the drug encapsulation capacity increased significantly, the hydrodynamic sizes of the corresponding KTZCFSMNPs stayed constant, remaining in the range 650 to 680 nm. The morphology and sizes of the KTZCFSMNPs were also examined by using transmission electron microscopy (TEM) and scan electron microscopy (SEM). Both TEM and SEM images suggested that the SMNPs exhibited spherical shapes with different sizes (FIG. 2c,d), finding that were consistent with those observed using DLS.

FIGS. 2A-2D are graphs and electron microscope images showing characterization of size-controlled ketoconazole-encapsulated cross-linked fluorescent supramolecular nanoparticles (KTZcFSMNPs). FIG. 2A shows dynamic light scattering data summarize the relationship between KTZcc-FSMNPs sizes and the mixing ratios of Ad-PAMAM/CD-PEI. FIG. 2B shows drug-encapsulation efficiency and capacity of KTZcFSMNPs with increasing drug loading concentration from 0.04 mg/mL to 0.4 mg/mL. High-performance liquid chromatography was used to test the concentration of KTZ. FIG. 2C shows transmission electron microscope images and FIG. 2D shows scanning electron microscope images of the resulting KTZcFSMNPs with the mixing ratios of the two molecular building blocks (Ad-PAMAM/CD-PEI) (i) 320±30 nm from 0.5/1, (ii) 440±30 nm from 1.5/1, (iii) 680±50 nm from 2.5/1.

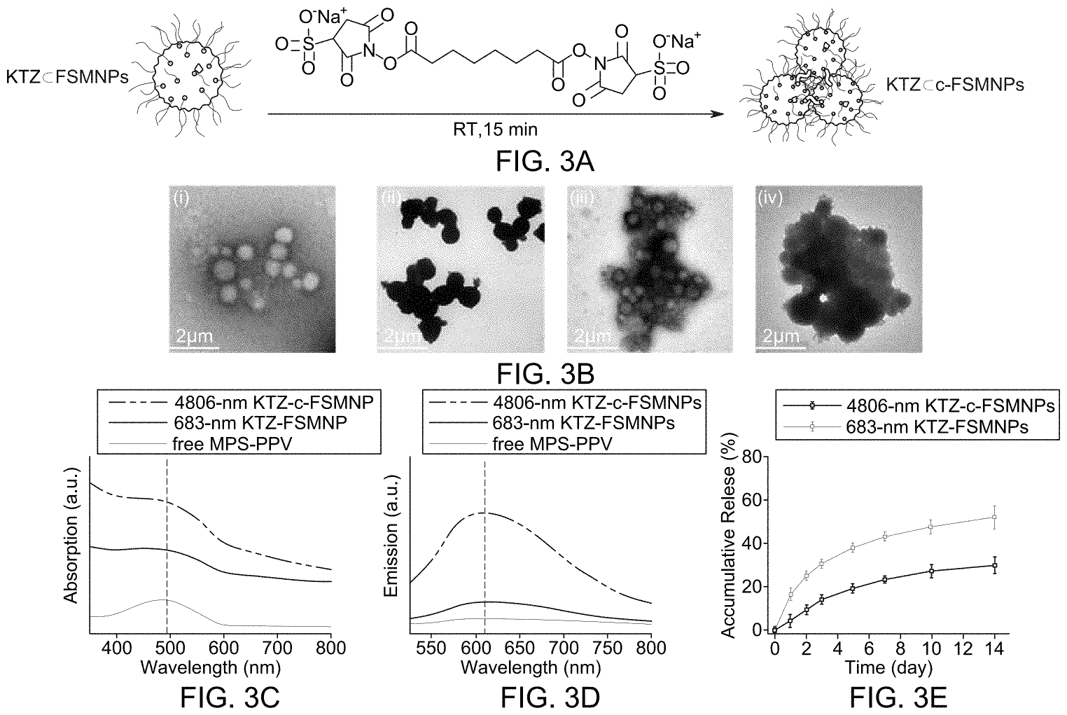

It was previously shown that, in the presence of a covalent amine-reactive cross-linker bis(sulfosuccinimidyl)suberate (BS3), FSMNP can be cross-linked to generate micrometer-sized c-FSMNPs with improved intradermal retention.³¹ On the basis of a similar synthetic procedure,³¹ a cross-linking reaction (FIG. 3a) to “glue” several 680 nm KTZCFSMNPs (with highest KTZ encapsulation capacity up to 9.4 wt %) together covalently was conducted. By altering the concentrations of BS3 (20, 40, 60, and 80 μg/mL) and keeping the concentration of KTZCFSMNPs constant (10 mg/mL), micrometer-sized KTZCc-FSMNPs were obtained with the hydrodynamic sizes of 2200±180, 3500±200, 4200±220, and 4800±230 nm. The micrometer-sized KTZCc-FSMNPs were characterized by TEM (FIG. 3b), confirming that KTZCc-FSMNPs were composed of 680 nm KTZCFSMNPs.

Knowing that 670 nm FSMNPs exhibit³¹ optimal fluorescent performance with 10-fold enhancement compared to that observed for free MPS-PPV, the photophysical properties of 4800 nm KTZCc-FSMNPs in comparison with the 680 nm KTZCFSMNPs and free MPS-PPV were examined. The 4800 nm KTZCc-FSMNPs showed enhanced absorption and fluorescence intensity, with 4.8-fold enhancement of 680 nm KTZCFSMNPs and 17-fold enhancement of free MPS-PPV (FIGS. 3c,d). This enhancement was largely attributable to the aggregate disassembly of MPS-PPV through the electrostatic interactions with CD-PEI in the KTZCc-FSMNPs. The drug releasing kinetics of 4800 nm KTZCc-FSMNPs and 680 nm KTZCFSMNPs (KTZ encapsulation capacities of both being 9.4 wt %) were monitored at 37° C. in 50% human serum (1:1 human serum: 1× PBS, v/v), under continuous and gentle shaking for 14 days. (FIG. 3e) As expected, the 4800 nm KTZCc-FSMNPs showed a more sustainable drug-release profile, with a release rate of 0.48 times that of 680 nm KTZCFSMNPs. The accumulated KTZ release of 4800 nm KTZCFSMNPs reached 30±4% after 14 days. From the difference in drug kinetics of the two systems, it was concluded that the covalent cross-linking played an important role in delaying the dynamic disassembly of KTZCc-FSMNPs by tightening the hydrogel networks. The KTZ was slowly released without any associated burst release, which avoided issues of systemic toxicity and insufficient local drug concentration.

FIGS. 3A-3E are schematics, images, and data graphs showing the formation and characterization of micrometer-sized ketoconazole-encapsulated cross-linked fluorescent supramolecular nanoparticles (KTZCc-FSMNPs). FIG. 3A shows BS3 as a cross-linker was introduced to the 680 nm KTZCFSMNPs solution to form micrometer-sized KTZCc-FSMNPs. FIG. 3B is transmission electron microscope images of the cross-linked KTZCFSMNPs with different sizes under the BS3 treatment with various concentrations: (i) (2200±180 nm) from 20 μg/mL, (ii) (3500±200 nm) from 40 μg/mL, (iii) (4200±220 nm) from 60 μg/mL, and (iv) (4800±230 nm) from 80 μg/mL. Comparison of (c) absorption and (d) emission spectra of free MPS-PPV, 680 nm KTZCFSMNPs and 4800 nm KTZCc-FSMNPs is shown in FIGS. 3C and 3D, respectively. FIG. 3E shows controlled release profiles by introducing 680 nm KTZCFSMNPs and 4800 nm KTZCc-FSMNPs with KTZ encapsulation capacities of 9.4 wt % at 37° C. in 50% human serum (human serum: PBS=1:1, v/v), under continuous and gentle shaking for 14 days. Released KTZ was quantified by HPLC.

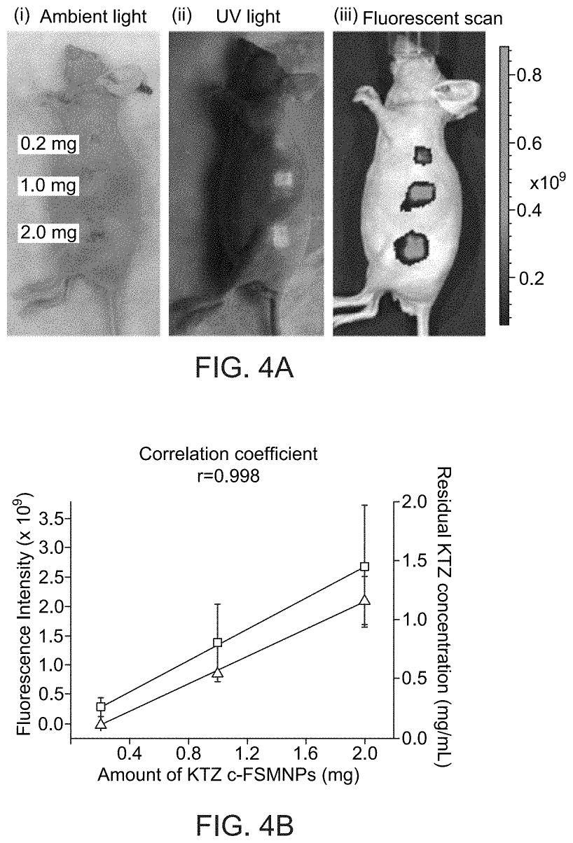

To study the in vivo properties of 4800 nm KTZCc-FSMNPs, the correlation between the fluorescent signals and residual KTZ concentrations of the 4800 nm KTZCc-FSMNPs in the mouse skin after tattoo deposition was studied. Three different amounts of KTZCc-FSMNPs (i.e., 0.2, 1.0, and 2.0 mg) were deposited at three adjacent locations on the skins of nu/nu mice (n=3) (FIG. 4a(i)). The strong fluorescent signals of the tattooed KTZCc-FSMNPs can be visualized by the naked eye under the irradiation of a UV lamp (365 nm; FIG. 4a(ii)) and quantified using in vivo optical imaging system (IVIS-200, PerkinElmer, excitation/emission, 570/620 nm; exposure time, 2 s; FIG. 4a(iii)). The mice were sacrificed and their tattooed skin tissues were harvested. After tissue homogenization and extraction by methanol, the KTZ in the mouse skin tissues were quantified by HPLC. As shown in FIG. 4b, the fluorescent intensities and residual KTZ showed strong linear relationships (correlation coefficient of 0.998) with the KTZCc-FSMNP quantities deposited via tattoo.

FIGS. 4A and 4B are images and a data graph showing the correlation between fluorescence intensity and residual ketoconazole (KTZ) concentration after tattoo deposition of 4800 nm KTZ-encapsulated cross-linked fluorescent supramolecular nanoparticles (KTZCc-FSMNPs). FIG. 4A shows three different amounts of KTZCc-FSMNPs (i.e., 0.2, 1.0, and 2.0 mg) are tattooed at three adjacent locations on the back of the nu/nu mice (n=3). (i) Photograph of a mouse tattooed with different amounts of KTZCc-FSMNPs under ambient light irradiation; (ii) image of the tattooed mouse under a UV irradiation (365 nm); (iii) fluorescent image of the tattooed mouse using in vivo optical imaging system (excitation/emission=570/620 nm; exposure time=2 s). FIG. 4B shows both fluorescence intensity and residual KTZ showed great linear relationships with the KTZCc-FSMNP quantities deposited via tattoo.

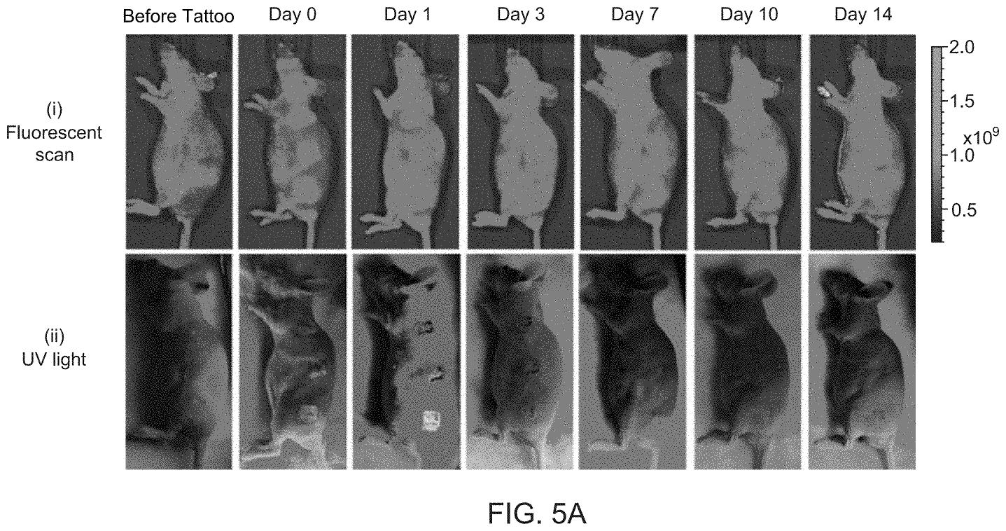

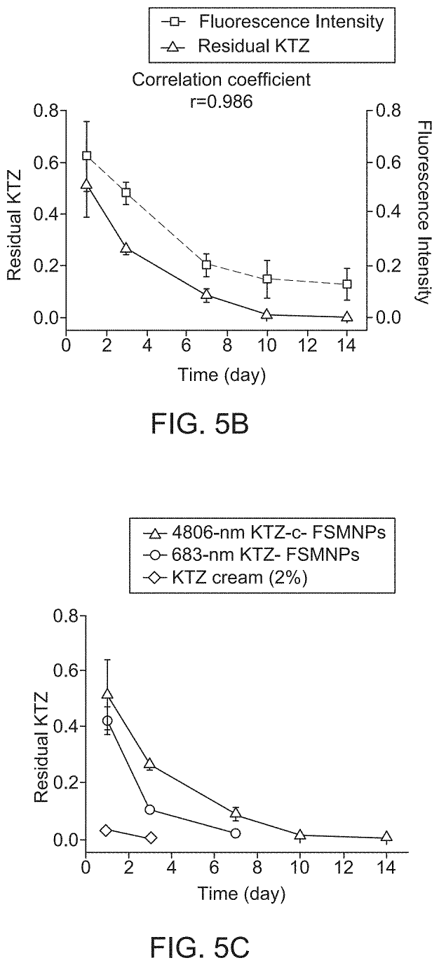

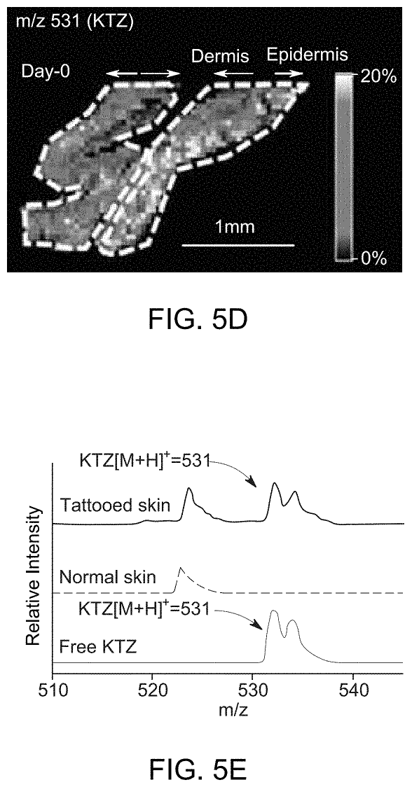

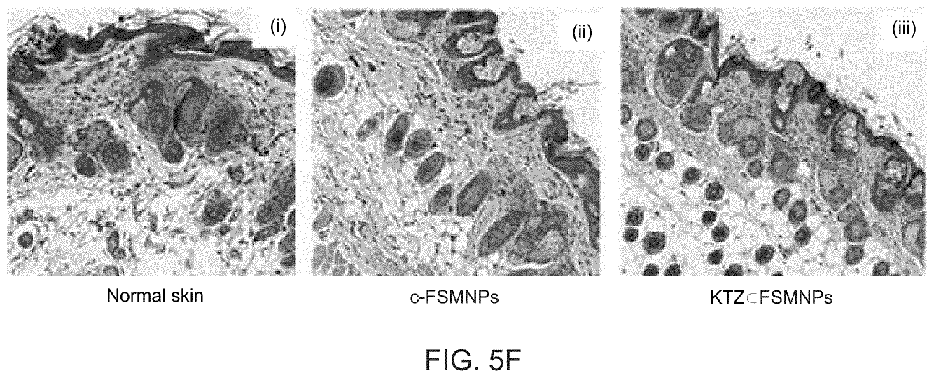

Since the fluorescence intensity and the residual KTZ correlate consistently, the presence of KTZCc-FSMNPs in the tattooed sites can be noninvasively monitored by their fluorescent signals. Time-dependent intradermal retention properties of 4800 nm KTZCc-FSMNPs were studied over a period of 14 days after their tattoo depositions, in which 2.0 mg of KTZCc-FSMNPs (equivalent to 200 μg of KTZ) were tattoo deposited at three adjacent locations (5 mm×5 mm) on the skins of nu/nu mice (n=6). An in vivo optical imaging system offers a great sensitivity for monitoring the fluorescent signals (excitation/emission: 570/620 nm; FIG. 5a(i)) of residual KTZCc-FSMNPs over a period of 14 days. In contrast, residual KTZCc-FSMNPs 7 days after tattoo deposition were invisible to naked eye observation under a UV lamp (365 nm) due to lower sensitivity (FIG. 5a(ii)). The mice were sacrificed at five different post-tattoo time points, and their tattooed skin tissues were harvested. After skin tissue homogenization and extraction by methanol, residual KTZ in skin tissues was quantified by HPLC, suggesting a finite intra-dermal retention time up to 14 days. In addition, the time-dependent fluorescent signals and residual KTZ were summarized in FIG. 5b, where fluorescent signals and KTZ quantities were normalized to the initial state at day 0. The gradual decay of fluorescent signals and decrease of KTZ quantities over time indicated the dynamic disassembly of KTZCc-FSMNPs under physiological condition. The high correlation coefficient (r=0.986) between fluorescent signals and KTZ quantities demonstrated that the intradermal retention of KTZ can be noninvasively monitored by the fluorescent signals of KTZCc-FSMNPs.

To illustrate the advantage of utilizing 4800 nm KTZCc-FSMNPs for intradermal delivery, tattoo deposition of 4800 nm KTZCc-FSMNPs, 680 nm KTZCFSMNPs, and topical treatment of KTZ cream (2%) on nu/nu mice skin, both equivalent to 200 μg of KTZ at each of the same area was carried out. After administration, the time-dependent KTZ decay in mouse skins was quantified using HPLC. As shown in FIG. 5c, 4800 nm KTZCc-FSMNPs showed the highest residual KTZ amounts and the slowest KTZ decay in the skins up 14 days. To test the presence of KTZ in intradermal tattooed skin, KTZ distribution in intradermal regions around the tattoo site after tattoo deposition of KTZcc-FSMNPs was mapped by MALDI-MSI.³⁹ The molecular ion of KTZ (chemical formula C₂₆H₂₉C₁₂N₄O₄; [M+H]⁺=m/z 531) was imaged in the day-0 (FIG. 5d) and day-3 tattooed longitudinal skin slices, which showed that KTZ was diffused throughout the intradermal region. Compared to tattooed skin, no obvious KTZ ([M+H⁺] at m/z 531) was detected in normal skin without KTZ⊂c-FSMNPs treatment (FIG. 5e). The pathological study of mouse skins was conducted at 14 days after tattoo depositions to validate the biocompatibility of KTZ⊂c-FSMNPs. The results of the H&E (hematoxylin, nucleus staining and eosin, cytoplasm staining) stained tissue sections were independently reviewed by our collaborator pathologist and dermatologist. Compared with normal skin and c-FSMNPs, no obvious inflammatory cells were observed in the H&E stained tissue sections tattooed with 4800 nm KTZ⊂c-FSMNPs at 14 days, indicating the biocompatibility of KTZ⊂c-FSMNPs.

FIGS. 5A-5F are images, data graphs, and stained skin sections from an intradermal retention study of ketoconazole-encapsulated cross-linked fluorescent supramolecular nanoparticles (KTZ⊂c-FSMNPs). In FIG. 5A a 2.0 mg sample of 4800 nm KTZ⊂c-FSMNPs (equivalent to 200 μg of KTZ) is tattooed at three adjacent locations (5 mm×5 mm) on the skins of nu/nu mice (n=6): (i) Fluorescent images of the tattooed mouse using in vivo optical imaging system (excitation/emission=465/520 nm; exposure time=2 s), for 14 days; (ii) images of the tattooed mouse under a UV light irradiation (365 nm), for 14 days. FIG. 5B shows time-dependent fluorescent signals and residual KTZ quantities of KTZ⊂c-FSMNPs in tattoo sites for 14 days. Both fluorescent signals and residual KTZ quantities were normalized to the initial measurements at day 0. FIG. 5C shows a comparison of time-dependent residual KTZ quantities in skins after tattoo depositions of KTZ⊂c-FSMNPs and KTZ⊂c-FSMNPs, as well as topical treatment of KTZ topical cream (2%). KTZ quantities were normalized to the initial ones at day 0. FIG. 5D shows direct detection of KTZ in intradermal region of the tattooed skin slices by MALDI-MSI. The ion images of KTZ (m/z=531) were acquired from two day-0 skin slices. FIG. 5E shows MALDI-MS spectra of tattooed skin slice, normal skin slice, and free KTZ. FIG. 5F shows H&E stained skin sections from a nu/nu mouse tattooed with 4800 nm KTZ⊂c-FSMNPs after 14 days after tattoo deposition (magnification: 100×). Compared to (i) normal skin without tattoo and (ii) c-FSMNPs without KTZ, no obvious inflammation cells were observed in the skin of nu/nu mouse after the tattoo-guided treatment of (iii) KTZcc-FSMNPs.

### Conclusion

In summary, successful preparation of KTZ⊂c-FSMNPs via a two-step synthetic approach, starting from supramolecular assembly of KTZ⊂c-FSMNPs from ratiometric mixing of antifungal drug (KTZ), a fluorescent reporter (MPS-PPV), and the three SMNP molecular building blocks, followed by cross-linking of KTZ⊂c-FSMNPs was shown. The sizes, encapsulation efficiency/capacity, photophysical properties, and KTZ controlled release profiles of the resulting KTZ⊂c-FSMNPs and KTZ⊂c-FSMNPs was characterized. Consequently, the 4800 nm KTZ⊂c-FSMNPs were chosen for in vivo studies using a mouse model, wherein the KTZ⊂c-FSMNPs were deposited intradermally via tattoo. Then, (i) in vivo fluorescence imaging was used to monitor the time-dependent fluorescence decay, (ii) HPLC was used to quantify residual KTZ in skin tissues, (iii) MALDI-MSI was used to map KTZ distribution in intradermal regions around the tattoo site, and (iv) histology was used to assess of local inflammatory responses and biocompatibility, to examine intradermal retention properties of 4800 nm KTZ⊂c-FSMNPs over a period of 14 days. The results presented herein constitute a proof-of-concept demonstration of 4800 nm KTZ⊂c-FSMNPs as an intradermal controlled release solution. This will allow minimally invasive, localized, and sustained delivery of therapeutic agents directly to the disease sites, maximizing the treatment efficacy of the drugs and avoiding the issues of systemic toxicity and insufficient local drug concentration. Further, it is conceivable that these c-FSMNP delivery vectors can be applied to treat a wide spectrum of clinically stubborn skin diseases that are in need of more efficient local drug concentration.

### MATERIALS AND METHODS

### Materials

Reagents and solvents were purchased from Sigma Aldrich (St. Louis, Mo.) and used as received without further purification unless otherwise mentioned. Branched polyethylenimine (PEI, MW=10 kD) was purchased from Polysciences, Inc. (Washington, Pa.). The polymers contain primary, secondary, and tertiary amine groups in approximately 25/50/25 ratio. First-generation polyamidoamine dendrimer (PAMAM) with 1,4-diaminobutane core and amine terminals in 20 wt % methanol solution was purchased from Andrews ChemServices, Inc (Berrien Springs, Mich.). 1-Adamantanamine (Ad) hydrochloride and β-cyclodextrin (β-CD) were purchased from TCI America (San Francisco, Calif.). N-hydroxysuccinimide (NETS) functionalized methoxyl polyethylene glycol (mPEG-NHS, MW=5 kD) was obtained from Creative PEGWorks, Inc (Chapel Hill, N.C.). 6-Mono-tosyl-β-cyclodextrin (6-OTs-β-CD) was prepared according to the literature reported method.⁴⁰ Octa-Ad-grafted polyamidoamine dendrimer (Ad-PAMAM), CD-grafted branched polyethylenimine (CD-PEI) and Ad-grafted polyethylene glycol (Ad-PEG) were synthesized by the method we previously reported.¹⁵ Poly[5-methoxy-2-(3-sulfopropoxy)-1,4-phenylenevinylene] potassium salt (MPS-PPV), ketoconazole, and diethylamine were purchased from Sigma-Aldrich (St. Louis, Mo.).

### Preparation of KTZ⊂c-FSMNPs

A self-assembly procedure was employed to achieve the ketoconazole-encapsulated fluorescent supramolecular nanoparticles (KTZ⊂c-FSMNPs). To a solution of Ad-PEG (1.84 mg/mL) in 485-μL of PBS buffer, CD-PEI (0.8 mg/mL) was slowly added under vigorous stirring at RT. MPS-PPV (0.12 mg/mL) was then added sequentially and the mixture solution was stirred vigorously for 2 min. Then, a 5-μL aliquot of DMSO containing Ad-PAMAM (0.4-2.0 mg/mL) and KTZ (0.04-0.4 mg/mL) was added into the mixture solution under vigorous stirring to obtain KTZ⊂c-FSMNPs.

### Preparation of KTZ⊂c-FSMNPs

The 680-nm sized KTZ⊂c-FSMNPs (10 mg/mL) were mixed with various concentrations of BS3 (20, 40, 60, 80, and 100 μg/mL) at RT with vigorous stirring. After 15 min, Tris buffer (1×) was added to the reaction solution in order to stop the cross-linking reaction of BS3.

### General Procedure for Tattoo Deposition of SMNPs

Prior to tattoo deposition, the skin of a nu/nu mouse was first wiped by alcohol prep pads twice. A 10 μL of KTZ⊂c-FSMNPs solution (containing 200 μg of pure KTZ) was introduced onto a unit area (5 mm×5 mm) of a nu/nu mouse skin (n=6). Immediately, a tattoo machine (Stingray Authentic X2 Rotary Tattoo Machine, InkMachines) was utilized to puncture (600 times per minute) over the designated area (5 mm×5 mm) in an average puncture depth of 0.2 mm. At 60 s after tattooing, the mouse skin was cleaned by alcohol prep pads twice to remove any residual KTZ⊂c-FSMNPs on the skin.

Correlation between Fluorescence Intensity and Residual KTZ Concentration after Tattoo Deposition of 4800 nm KTZ⊂c-FSMNPs

All animal manipulations were performed with sterile technique and were approved by the Institutional Animal Care and Use Committee of University of Southern California. Female athymic nude mice (about 6-8 weeks old, with a body weight of 20-25 g) were purchased from Envigo (Livermore, Calif., USA). After the mice were anesthetized with 2% isoflurane in a heated (37° C.) induction chamber, mouse skin was poked with a commercial tattoo device to make wounds to the dermal layer. After tattoo depositions, the signals of KTZ⊂c-FSMNPs were measured with the in vivo optical imaging system (IVIS-200, PerkinElmer, Waltham, Mass., USA). The mice were sacrificed, and their tattooed skin tissues were harvested. After tissue homogenization and extraction by methanol, the extracts were vortexed twice for 15 s and centrifuged at 10000 rpm for 10 min. The KTZ-containing supernatants were filtrated through 0.22 μm filters for HPLC analysis at a flow rate of 1 mL/min. The statistical analysis of the correlation between the fluorescent intensity and residual KTZ concentration was performed using a correlation analysis (GraphPad Prism 6.0).

### Drug Loading Efficiency of KTZ⊂c-FSMNPs or KTZ⊂c-FSMNPs

A collection of 683-nm sized KTZ⊂FSMNPs or 4806-nm sized KTZ⊂c-FSMNPs with various loading concentration of KTZ (0.04-0.4 mg/mL) was obtained according to the above protocol. Typically, non-encapsulated KTZ was removed from KTZ⊂c-FSMNPs by centrifugation of KTZ⊂c-FSMNPs solution at 1300 rpm for 30 min using centrifugal filter devices (3000 NMWL). After recovering the filtrate containing non-encapsulated KTZ, the KTZ concentration was analyzed by high-performance liquid chromatography (HPLC) in a system equipped with a Knauer Smartline pneumatic pump, C18 column, K-2600 spectrophotometer, and Gina data acquisition software. A mixture of acetonitrile and water (containing 0.05% diethylamine) at a volume ratio of 7:3 was used as the mobile phase. The flow rate was set at 1 mL/min. 25 μL of KTZ-containing sample was injected to measure the drug absorption at 227 nm, typically eluted in 4.3 min. The measurements were performed in triplicate. The amount of the KTZ encapsulated into KTZ⊂c-FSMNPs was then calculated by subtracting the free KTZ in the filtrate from the total loading amount of KTZ. Drug loading efficiency (DLE) of the resulting KTZ⊂c-FSMNPs was obtained as the amount of the KTZ encapsulated in the KTZ⊂c-FSMNPs vector divided by the total loading amount of KTZ.

Drug Release from KTZ⊂FSMNPs or KTZ⊂c-FSMNPs

The 683-nm sized KTZ⊂FSMNPs (10 mg) or 4806-nm sized KTZ⊂c-FSMNPs (10 mg) was dispersed in 10 mL of 50% human serum (human serum: 1×PBS=1:1, v/v), the dispersion was divided into five equal aliquots. Each of the aliquot sample was then transferred into a dialysis tubing (molecular weight cut off 14,000), which was dialyzed against 18 mL of the 50% human serum, and then incubated at 37° C. under continuous and gentle shaking for 14 days. At selected time intervals, 1 mL of the solution was obtained periodically from the reservoir, and the amount of released KTZ was analyzed by HPLC equipped with an analytical C18 column. The area of the HPLC peak of the released KTZ was intergraded for the quantification of KTZ as compared to a calibration curve of free KTZ prepared separately.

### Examination on Intradermal Retention Time of KTZ⊂c-FSMNPs

Similarly, 2.0 mg of KTZ⊂c-FSMNPs (equivalent to 200 μg of KTZ) were tattoo deposited at three adjacent locations (5 mm×5 mm) on the skins of nu/nu mice (n=6). After tattoo depositions, the signals of KTZ⊂c-FSMNPs were measured with the in vivo optical imaging system (IVIS-200, PerkinElmer, Waltham, Mass., USA) at selected time intervals prolonged for 14 days. The tissue samples on tattoo sites were also collected and homogenized on ice, followed by the extraction of tissue homogenate in 0.5 mL of methanol and quantification of the residual KTZ in mouse skin through HPLC analysis to evaluate the drug decay in 14 days. To compare the intradermal retention time with non-cross-linked KTZ⊂FSMNPs and KTZ cream (2%), the tattoo-guided treatment of 680 nm KTZcFSMNPs and topical treatment of KTZ cream (2%) were conducted by applying an equivalent to 200 μg of KTZ at each of the same areas and then quantifying the extracted drug concentrations of application sites.

Pathological Studies of Skin Tissues Tattooed with KTZ⊂c-FSMNPs

Skin tissues were taken from another group of mice, treated the same as described above in the in vivo study, 14 days after tattooing for pathological studies. Skin tissues were fixed with 10% formalin and blocked with paraffin, following conventional laboratory methods. Slices of skin tissue were stained with H&E (Hematoxylin and eosin) solution for pathological study. Tissues were then examined using an Aperio ScanScope AT microscope (Leica biosystem, USA). Each H&E stained tissue slide was evaluated by two independent pathologists.

The embodiments illustrated and discussed in this specification are intended only to teach those skilled in the art how to make and use the invention. In describing embodiments of the invention, specific terminology is employed for the sake of clarity. However, the invention is not intended to be limited to the specific terminology so selected. The above-described embodiments of the invention may be modified or varied, without departing from the invention, as appreciated by those skilled in the art in light of the above teachings. Moreover, features described in connection with one embodiment of the invention may be used in conjunction with other embodiments, even if not explicitly stated above. It is therefore to be understood that, within the scope of the claims and their equivalents, the invention may be practiced otherwise than as specifically described.

### REFERENCES

(1) Gupta, A. K.; Drummond-Main, C.; Cooper, E. A.; Brintnell, W.; Piraccini, B. M.; Tosti, A. Systematic Review of Nondermatophyte Mold Onychomycosis: Diagnosis, Clinical Types, Epidemiology, and Treatment. J. Am. Acad Dermatol. 2012, 66, 494-502.
(2) Gupta, A. K.; Studholme, C. Novel Investigational Therapies for Onychomycosis: An Update. Expert Opin. Invest. Drugs 2016, 25, 297-305.
(3) Sigurgeirsson, B.; Baran, R. The Prevalence of Onychomycosis in The Global Population—A Literature Study. J. Eur. Acad Dermatol. Venereol. 2014, 28, 1480-1491.
(4) Rosen, T.; Friedlander, S. F.; Kircik, L.; Zirwas, M. J.; Stein, L. G.; Bhatia, N.; Gupta, A. K. Onychomycosis: Epidemiology, Diagnosis, and Treatment in a Changing Landscape. J. Drugs Dermatol. 2015, 14, 223-233.
(5) Lima, A. L.; Illing, T.; Schliemann, S.; Elsner, P. Cutaneous Manifestations of Diabetes Mellitus: A Review. Am. J. Clin. Dermatol. 2017, 18, 541-553.
(6) Thomas, J.; Jacobson, G. A.; Narkowicz, C. K.; Peterson, G. M.; Burnet, H.; Sharpe, C. Toenail Onychomycosis: An Important Global Disease Burden. J. Clin. Pharm. Ther. 2010, 35, 497-519.
(7) Roujeau, J. C.; Sigurgeirsson, B.; Korting, H. C.; Kerl, H.; Paul, C. Chronic Dermatomycoses of The Foot As Risk Factors for Acute Bacterial Cellulitis of The Leg: A Case-Control Study. Dermatology 2004, 209, 301-307.
(8) Drake, L. A.; Patrick, D. L.; Fleckman, P.; Andre, J.; Baran, R.; Haneke, E.; Sapede, C.; Tosti, A. The Impact of Onychomycosis on Quality of Life: Development of an International Onychomycosis-Specific Questionnaire to Measure Patient Quality of Life. J. Am. Acad. Dermatol. 1999, 41, 189-196.
(9) Barot, B. S.; Parejiya, P. B.; Patel, H. K.; Mehta, D. M.; Shelat, P. K. Drug Delivery to the Nail: Therapeutic Options and Challenges for Onychomycosis. Crit. Rev. Ther. Drug Carrier Syst. 2014, 31, 459-494.
(10) Kushwaha, A.; Murthy, R. N.; Murthy, S. N.; Elkeeb, R.; Hui, X.; Maibach, H. I. Emerging Therapies for the Treatment of Ungual Onychomycosis. Drug Dev. Ind. Pharm. 2015, 41, 1575-1581.
(11) Gupta, A.; Simpson, F. Device-Based Therapies for Onychomy-cosis Treatment. Skin Therapy Lett. 2012, 17, 4-9.
(12) Sugiura, K.; Sugimoto, N.; Hosaka, S.; Katafuchi-Nagashima, M.; Arakawa, Y.; Tatsumi, Y.; Siu, W. J.; Pillai, R. The Low Keratin Affinity of Efinaconazole Contributes to Its Nail Penetration and Fungicidal Activity in Topical Onychomycosis Treatment. Antimicrob. Agents Chemother. 2014, 58, 3837-3842.
(13) Gupta, A. K.; Simpson, F. C. New Pharmacotherapy for the Treatment of Onychomycosis: An Update. Expert Opin. Pharmacother. 2015, 16, 227-236.
(14) Arrese, J. E.; Pierard, G. E. Treatment Failures and Relapses in Onychomycosis: A Stubborn Clinical Problem. Dermatology 2003, 207, 255-260.
(15) Gupta, A. K.; Daigle, D.; Foley, K. A. Topical Therapy for Toenail Onychomycosis: An Evidence-Based Review. Am. J. Clin. Dermatol. 2014, 15, 489-502.
(16) Saner, M. V.; Kulkarni, A. D.; Pardeshi, C. V. Insights into Drug Delivery Across the Nail Plate Barrier. J. Drug Target. 2014, 22, 769-789.
(17) McAuley, W. J.; Jones, S. A.; Traynor, M. J.; Guesne, S.; Murdan, S.; Brown, M. B. An Investigation of How Fungal Infection Influences Drug Penetration through Onychomycosis Patient's Nail Plates. Eur. J. Pharm. Biopharm. 2016, 102, 178-184.
(18) Bhatta, A. K.; Keyal, U.; Huang, X.; Zhao, J. J. Fractional Carbon-Dioxide (CO2) Laser-Assisted Topical Therapy for the Treatment of Onychomycosis. J. Am. Acad. Dermatol. 2016, 74, 916-923.
(19) Carney, C.; Cantrell, W.; Warner, J.; Elewski, B. Treatment of Onychomycosis Using A Submillisecond 1064-nm Neodymium:Yt-trium-Aluminum-Garnet Laser. J. Am. Acad. Dermatol. 2013, 69, 578-582.
(20) Wang, H.; Wang, S.; Su, H.; Chen, K. J.; Armijo, A. L.; Lin, W. Y.; Wang, Y.; Sun, J.; Kamei, K.; Czernin, J.; Radu, C. G.; Tseng, H. R. A Supramolecular Approach for Preparation of Size-Controlled Nano-particles. Angew. Chem., Int. Ed. 2009, 48, 4344-4348.
(21) Wang, S.; Chen, K.-J.; Wu, T.-H.; Wang, H.; Lin, W.-Y.; Ohashi, M.; Chiou, P.-Y.; Tseng, H.-R. Photothermal Effects of Supra-molecularly Assembled Gold Nanoparticles for the Targeted Treatment of Cancer Cells. Angew. Chem., Int. Ed. 2010, 49, 3777-3781.
(22) Wang, H.; Liu, K.; Chen, K.-J.; Lu, Y.; Wang, S.; Lin, W.-Y.; Guo, F.; Kamei, K.-i.; Chen, Y.-C.; Ohashi, M.; et al. A Rapid Pathway toward A Superb Gene Delivery System: Programming Structural and Functional Diversity into A Supramolecular Nanoparticle Library. ACS Nano 2010, 4, 6235-6243.
(23) Wang, H.; Chen, K.-J.; Wang, S.; Ohashi, M.; Kamei, K.-i.; Sun, J.; Ha, J. H.; Liu, K.; Tseng, H.-R. A Small Library of DNA-Encapsulated Supramolecular Nanoparticles for Targeted Gene Delivery. Chem. Commun. 2010, 46, 1851-1853.
(24) Chen, K.-J.; Wolahan, S. M.; Wang, H.; Hsu, C.-H.; Chang, H.-W.; Durazo, A.; Hwang, L.-P.; Garcia, M. A.; Jiang, Z. K.; Wu, L.; et al. A Small MRI Contrast Agent Library of Gadolinium (III)-Encapsulated Supramolecular Nanoparticles for Improved Relaxivity and Sensitivity. Biomaterials 2011, 32, 2160-2165.
(25) Liu, Y.; Wang, H.; Kamei, K. i.; Yan, M.; Chen, K. J.; Yuan, Q.; Shi, L.; Lu, Y.; Tseng, H. R. Delivery of Intact Transcription Factor by Using Self-Assembled Supramolecular Nanoparticles. Angew. Chem., Int. Ed. 2011, 123, 3114-3118.
(26) Chen, K.-J.; Tang, L.; Garcia, M. A.; Wang, H.; Lu, H.; Lin, W.-Y.; Hou, S.; Yin, Q.; Shen, C. K.-F.; Cheng, J.; Tseng, H.-R. The Therapeutic Efficacy of Camptothecin-Encapsulated Supramolecular Nanoparticles. Biomaterials 2012, 33, 1162-1169.
(27) Peng, J.; Garcia, M. A.; Choi, J.-s.; Zhao, L.; Chen, K.-J.; Bernstein, J. R.; Peyda, P.; Hsiao, Y.-S.; Liu, K. W.; Lin, W.-Y.; et al. Molecular Recognition Enables Nanosubstrate-Mediated Delivery of Gene-Encapsulated Nanoparticles with High Efficiency. ACS Nano 2014, 8, 4621-4629.
(28) Hou, S.; Choi, J. s.; Chen, K. J.; Zhang, Y.; Peng, J.; Garcia, M. A.; Yu, J. H.; Thakore-Shah, K.; Ro, T.; Chen, J. F.; et al. Supramolecular Nanosubstrate-Mediated Delivery for Reprogramming and Trans-differentiation of Mammalian Cells. Small 2015, 11, 2499-2504.
(29) Liu, Y.; Du, J.; Choi, J. s.; Chen, K. J.; Hou, S.; Yan, M.; Lin, W. Y.; Chen, K. S.; Ro, T.; Lipshutz, G. S.; et al. A High-Throughput Platform for Formulating and Screening Multifunctional Nanoparticles Capable of Simultaneous Delivery of Genes and Transcription Factors. Angew. Chem., Int. Ed. 2016, 55, 169-173.
(30) Lee, J. H.; Chen, K. J.; Noh, S. H.; Garcia, M. A.; Wang, H.; Lin, W. Y.; Jeong, H.; Kong, B. J.; Stout, D. B.; Cheon, J.; Tseng, H. R. On-Demand Drug Release System for In Vivo Cancer Treatment through Self-Assembled Magnetic Nanoparticles. Angew. Chem., Int. Ed. 2013, 52, 4384-8.
(31) Choi, J. S.; Zhu, Y. Z.; Li, H.; Peyda, P.; Nguyen, T. T.; Shen, M. Y.; Yang, Y. M.; Zhu, J. Y.; Liu, M.; Lee, M. M.; Sun, S. S.; Yang, Y.; Yu, H. H.; Chen, K.; Chuang, G. S.; Tseng, H. R. Cross-Linked Fluorescent Supramolecular Nanoparticles as Finite Tattoo Pigments with Controllable Intradermal Retention Times. ACS Nano 2017, 11, 153-162.
(32) Deng, P. Z.; Teng, F. F.; Zhou, F. L.; Song, Z. M.; Meng, N.; Liu, N.; Feng, R. L. Y-Shaped Methoxy Poly(Ethylene Glycol)-Block-Poly(Epsilon-Caprolactone)-Based Micelles for Skin Delivery of Ketoconazole: In Vitro Study and In Vivo Evaluation. Mater. Sci. Eng., C 2017, 78, 296-304.
(33) Ma, G.; Wu, C. Microneedle, Bio-Microneedle and Bio-Inspired Microneedle: A Review. J. Controlled Release 2017, 251,11-23.
(34) Bhatnagar, S.; Dave, K.; Venuganti, V. V. K. Microneedles in the Clinic. J. Control. Release 2017, 260, 164-182.
(35) Arya, J.; Henry, S.; Kalluri, H.; McAllister, D. V.; Pewin, W. P.; Prausnitz, M. R. Tolerability, Usability and Acceptability of Dissolving Microneedle Patch Administration in Human Subj ects. Biomaterials 2017, 128, 1-7.
(36) Yu, J.; Zhang, Y.; Ye, Y.; DiSanto, R.; Sun, W.; Ranson, D.; Ligler, F. S.; Buse, J. B.; Gu, Z. Microneedle-Array Patches Loaded with Hypoxia-Sensitive Vesicles Provide Fast Glucose-Responsive Insulin Delivery. Proc. Natl. Acad Sci. U.S.A. 2015, 112, 8260-8265.
(37) Wang, C.; Ye, Y.; Hochu, G. M.; Sadeghifar, H.; Gu, Z. Enhanced Cancer Immunotherapy by Microneedle Patch-Assisted Delivery of Anti-PD1 Antibody. Nano Lett. 2016, 16, 2334-2340.
(38) Di, J.; Yao, S.; Ye, Y.; Cui, Z.; Yu, J.; Ghosh, T. K.; Zhu, Y.; Gu, Z. Stretch-Triggered Drug Delivery from Wearable Elastomer Films Containing Therapeutic Depots. ACS Nano 2015, 9, 9407-9415.
(39) Lin, L.-E.; Su, P.-R.; Wu, H.-Y.; Hsu, C.-C. A Simple Sonication Improves Protein Signal in Matrix-Assisted Laser Desorption Ionization Imaging. J. Am. Soc. Mass Spectrom. 2018, 29, 1-4.
(40) Petter, R. C.; Salek, J. S.; Sikorski, C. T.; Kumaravel, G.; Lin, F. T. Cooperative Binding by Aggregated Mono-6-(Alkylamino)-Beta-Cyclo-dextrins. J. Am. Chem. Soc. 1990, 112, 3860-3868.

We claim:

1. A composition for delivering a drug to a subject comprising:
a plurality of self-assembled supramolecular nanoparticles (SMNPs), each of the plurality of self-assembled supramolecular nanoparticles (SMNPs) comprising:
a plurality of binding components, each having a plurality of binding regions;
a plurality of cores that are suitable to at least provide some mechanical structure to the plurality of self-assembled supramolecular nanoparticles (SMNPs), the plurality of cores comprising at least one core binding element adapted to bind to the binding regions to form a first inclusion complex;
a plurality of terminating components, each having a single terminating binding element that binds to remaining binding regions of one of said plurality of binding components by forming a second inclusion complex;
the drug; and
a reporter agent,
wherein the plurality of binding components and the plurality of cores self-assemble when brought into contact to form the plurality of self-assembled supramolecular nanoparticles (SMNPs),
wherein the plurality of terminating components act to occupy the remaining binding regions of the plurality of binding components, and the plurality of terminating components are present in a sufficient quantity relative to the plurality of binding regions of the plurality of binding components to terminate further binding, thereby forming a discrete particle,
wherein the drug is encapsulated within each of the plurality of self-assembled supramolecular nanoparticles (SMNPs),
wherein the reporter agent is encapsulated within each of the plurality of supramolecular nanoparticles (SMNPs),
wherein the plurality of cores and the plurality of binding components are present in a percent mass (w/w) ratio of between 0.25:1 and 2.5:1, and
wherein each of the plurality of self-assembled supramolecular nanoparticles (SMNPs) has a diameter of between 240 nanometers and 730 nanometers.

2. The composition of claim 1, wherein the plurality of binding components comprises polythylenimine, poly(L-lysine), or poly(β-amino ester).

3. The composition of claim 1, wherein the plurality of binding regions comprises beta-cyclodextrin, alpha-cyclodextrin, gamma-cyclodextrin, cucurbituril or calixarene.

4. The composition of claim 1, wherein the plurality of cores comprises polyamidoamine dendrimers, poly(prophylenimine) (PPI) dendrimer, triazine dendrimer, carbosilane dendrimer, poly(ether imine) (PETIM) dendrimer or phosphorus dendrimer.

5. The composition of claim 1, wherein the at least one core binding element comprises adamantanamine, azobenzene, ferrocene or anthracene.

6. The composition of claim 1, wherein the plurality of terminating components comprises polyethylene glycol (PEG) or poly(propylene glycol) (PGG).

7. The composition of claim 1, wherein the single terminating binding element comprises adamantanamine, azobenzene, ferrocene or anthracene.

8. The composition of claim 1, wherein the plurality of self-assembled supramolecular nanoparticles (SMNPs) are configured to release the drug into the subject over a period of time.

9. The composition of claim 8, wherein the period of time is at least 14 days in length.

10. The composition of claim 1, wherein each of the plurality of SMNPs are cross-linked to one or more of the plurality of SMNPs such that a cross-linked network of SMNPs is formed.

11. The composition of claim 10, wherein the cross-linked network of SMNPs has a maximum spatial dimension of between 2020 nanometers and 5030 nanometers.

12. The composition of claim 1, wherein the drug is selected from the group consisting of an anti-viral drug, an anti-bacterial drug, and an anti-fungal drug.

13. The composition of claim 1, wherein the reporter agent is a fluorescent probe.

14. The composition of claim 1, wherein the plurality of self-assembled supramolecular nanoparticles (SMNPs) are configured to release the drug at a first rate of release,
wherein the plurality of self-assembled supramolecular nanoparticles (SMNPs) are configured to release the reporter agent at a second rate of release, and
wherein the second rate of release is correlated with first rate of release.

15. A method for making a composition comprising a plurality of self-assembled supramolecular nanoparticles (SMNPs) for delivering a drug to a subject comprising:
providing a first solution comprising a plurality of binding components, each having a plurality of binding regions;
providing a second solution comprising a plurality of cores that are suitable to at least provide some mechanical structure to the plurality of self-assembled supramolecular nanoparticles (SMNPs), the plurality of cores comprising at least one core binding element adapted to bind to the binding regions to form a first inclusion complex;
providing a third solution comprising a plurality of terminating components, each having a single terminating binding element that binds to remaining binding regions of one of said plurality of binding components by forming a second inclusion complex;
providing a fourth solution comprising the drug;
providing a fifth solution comprising a reporter agent; and
mixing the first solution, the second solution, the third solution, the fourth solution, and the fifth solution,
wherein the mixing brings into contact the plurality of binding components and the plurality of cores such that the plurality of binding components and the plurality of cores self-assemble to form the plurality of self-assembled supramolecular nanoparticles (SMNPs), and such that the drug and the reporter agent are encapsulated within each of the plurality of self-assembled supramolecular nanoparticles (SMNPs),
wherein the plurality of terminating components act to occupy the remaining binding regions of the plurality of binding components, and the plurality of terminating components are present in a sufficient quantity relative to the plurality of binding regions of the plurality of binding components to terminate further binding, thereby forming a discrete particle,
wherein the wherein the plurality of cores and the plurality of binding components are present in a percent mass (w/w) ratio of between 0.25:1 and 2.5:1, and
wherein each of the plurality of self-assembled supramolecular nanoparticles (SMNPs) has a diameter of between 240 nanometers and 730 nanometers.

16. The method of claim 15, wherein the plurality of binding components comprises polythylenimine, poly(L-lysine) or poly(β-amino ester).

17. The method of claim 15, wherein the plurality of binding regions comprises beta-cyclodextrin, alpha-cyclodextrin, gamma-cyclodextrin, cucurbituril or calixarene.

18. The method of claim 15, wherein the plurality of cores comprises polyamidoamine dendrimers, poly(prophylenimine) (PPI) dendrimer, triazine dendrimer, carbosilane dendrimer, poly(ether imine) (PETIM) dendrimer or phosphorus dendrimer.

19. The method of claim 15, wherein the at least one core binding element comprises adamantanamine, azobenzene, ferrocene or anthracene.

20. The method of claim 15, wherein the plurality of terminating components comprises polyethylene glycol (PEG) or poly(propylene glycol) (PGG).

21. The method of claim 15, wherein the single terminating binding element comprises adamantanamine, azobenzene, ferrocene or anthracene.

22. The method of claim 15, wherein the plurality of self-assembled supramolecular nanoparticles (SMNPs) are configured to release the drug into the subject over a period of time.

23. The method of claim 22, wherein the period of time is at least 14 days in length.

24. The method of claim 15, further comprising cross-linking the each of the plurality of self-assembled supramolecular nanoparticles (SMNPs) to one or more of the plurality of self-assembled supramolecular nanoparticles (SMNPs) such that a cross-linked network of self-assembled supramolecular nanoparticles (SMNPs) is formed.

25. The method of claim 24, wherein the cross-linked network of self-assembled supramolecular nanoparticles (SMNPs) has a maximum spatial dimension of between 2020 nanometers and 5030 nanometers.

26. The method of claim 15, wherein the drug is selected from the group consisting of an anti-viral drug, an anti-bacterial drug, and an anti-fungal drug.

27. The method of claim 15, wherein the reporter agent is a fluorescent probe.

28. The method of claim 15, wherein the plurality of self-assembled supramolecular nanoparticles (SMNPs) are configured to release the drug at a first rate of release,
wherein the plurality of self-assembled supramolecular nanoparticles (SMNPs) are configured to release the reporter agent at a second rate of release, and
wherein the second rate of release is correlated with first rate of release.

29. A method for delivering a drug to a subject comprising:
penetrating an epidermis tissue layer of the subject such that an accession point to an underlying dermis layer in the subject is created; and
delivering a plurality of self-assembled supramolecular nanoparticles (SMNPs) to the underlying dermis layer in the subject through the accession point,
wherein each of the plurality of self-assembled supramolecular nanoparticles (SMNPs) comprises:
a plurality of binding components, each having a plurality of binding regions;
a plurality of cores that are suitable to at least provide some mechanical structure to the plurality of self-assembled supramolecular nanoparticles (SMNPs), the plurality of cores comprising at least one core binding element adapted to bind to the binding regions to form a first inclusion complex;
a plurality of terminating components, each having a single terminating binding element that binds to remaining binding regions of one of said plurality of binding components by forming a second inclusion complex;
the drug; and
a reporter agent,
wherein the plurality of binding components and the plurality of cores self-assemble when brought into contact to form the plurality of self-assembled supramolecular nanoparticles (SMNPs),
wherein the plurality of terminating components act to occupy the remaining binding regions of the plurality of binding components, and the plurality of terminating components are present in a sufficient quantity relative to the plurality of binding regions of the plurality of binding components to terminate further binding, thereby forming a discrete particle,
wherein the drug is encapsulated within each of the plurality of self-assembled supramolecular nanoparticles (SMNPs),
wherein the reporter agent is encapsulated within each of the plurality of supramolecular nanoparticles (SMNPs),
wherein the wherein the plurality of cores and the plurality of binding components are present in a percent mass (w/w) ratio of between 0.25:1 and 2.5:1, and
wherein each of the plurality of self-assembled supramolecular nanoparticles (SMNPs) has a diameter of between 240 nanometers and 730 nanometers.

30. The method of claim 29, wherein the plurality of binding components comprises polythylenimine, poly(L-lysine), or poly(β-amino ester).

31. The method of claim 29, wherein the plurality of binding regions comprises beta-cyclodextrin, alpha-cyclodextrin, gamma-cyclodextrin, cucurbituril or calixarene.

32. The method of claim 29, wherein the plurality of cores comprises polyamidoamine dendrimers, poly(prophylenimine) (PPI) dendrimer, triazine dendrimer, carbosilane dendrimer, poly(ether imine) (PETIM) dendrimer or phosphorus dendrimer.

33. The method of claim 29, wherein the at least one core binding element comprises adamantanamine, azobenzene, ferrocene or anthracene.

34. The method of claim 29, wherein the plurality of terminating components comprises polyethylene glycol (PEG) or poly(propylene glycol) (PGG).

35. The method of claim 29, wherein the single terminating binding element comprises adamantanamine, azobenzene, ferrocene or anthracene.

36. The method of claim 29, wherein the plurality of self-assembled supramolecular nanoparticles (SMNPs) are configured to release the drug into the subject over a period of time.

37. The method of claim 29, wherein the period of time is at least 14 days in length.

38. The method of claim 29, wherein each of the plurality of self-assembled SMNPs are cross-linked to one or more of the plurality of self-assembled SMNPs such that a cross-linked network of SMNPs is formed.

39. The method of claim 38, wherein the cross-linked network of SMNPs has a maximum spatial dimension of between 2020 nanometers and 5030 nanometers.

40. The method of claim 29, wherein the drug is selected from the group consisting of an anti-viral drug, an anti-bacterial drug, and an anti-fungal drug.

41. The method of claim 29, wherein the reporter agent is a fluorescent probe.

42. The method of claim 29, wherein the plurality of self-assembled supramolecular nanoparticles (SMNPs) are configured to release the drug at a first rate of release,
wherein the plurality of self-assembled supramolecular nanoparticles (SMNPs) are configured to release the reporter agent at a second rate of release, and
wherein the second rate of release is correlated with first rate of release.
